(19) United States
(12) Patent Application Publication
(10) Pub. No.: US 2010/0272740 A1
(43) Pub. Date: Oct. 28, 2010

(54) MICRO- AND NANOSCALE DEVICES FOR DELIVERY OF ACTIVE FIBRONOLYTIC AGENTS

(76) Inventors: Alexey Vertegel, Easley, SC (US); Yuliya Yurko, Easley, SC (US)

Correspondence Address: DORITY & MANNING, P.A., POST OFFICE BOX 1449, GREENVILLE, SC, 29602-1449, US

(21) Appl. No.: 12/675,977

(22) PCT Filed: Oct. 17, 2008
(86) PCT No.: PCT/US08/80295

§ 371 Date: Jun. 21, 2010

Related U.S. Application Data
(60) Provisional application No. 60/980,614, filed on Oct. 17, 2007.

(51) Int. Cl. A61K 39/395; C07K 16/18; A61P 7/00

(52) U.S. Cl. 424/178.1; 530/391.7

(57) ABSTRACT

Disclosed are devices as may be used to deliver a fibrinolytic agent such as functional tPA to targeted sites. Disclosed devices include micro or nanosized particles as carriers of a fibrinolytic agent and a targeting polypeptide such as antifibrin antibody that specifically binds a component of a blood clot. A plurality of protein molecules can be bound to a single particle. In addition, the total number of protein molecules attached to each particle can be controlled as can the proportion of each different compound bound to a single particle. Disclosed devices can be utilized, for example, to deliver tPA to blood clots, for instance in postmyocardial infarction or ischemic stroke treatment, and can minimize systemic plasminemia compared to the use of free tPA.

### CROSS REFERENCE TO RELATED APPLICATIONS

The present application claims filing benefit of U.S. Provisional Application Ser. No. 60/980,614 having a filing date of Oct. 17, 2007, which is incorporated herein in its entirety.

### FEDERALLY SPONSORED RESEARCH AND DEVELOPMENT

The United States Government may have rights in this disclosure pursuant to National Science Foundation Grant No. CMS-0619739.

### BACKGROUND

The ability to break down fibrin containing structures such as blood clots plays a crucial role in many treatment processes. For instance, tissue plasminogen activator (tPA) is a serine protease fibrinolytic agent that primarily functions as a proteolytic activator of the proenzyme plasminogen to form plasmin. Plasmin is a fibrinolytic enzyme that acts upon fibrin and can break down blood clots. Currently, recombinant tPA is used clinically in postmyocardial infarction and ischemic stroke for recanalization and reperfusion. Unfortunately, tPA also acts upon plasminogen in circulation generating systemic plasmin and rendering the treated patient highly vulnerable to hemorrhage. In fact, administration of recombinant tPA has been associated with a high patient mortality rate (6.3% for 30-day mortality) including fatal cerebral hemorrhage in 1.5% of patients. While beneficial effects of tPA-based thrombolytic therapy have clearly been established, a decrease in the associated mortality rate would vastly improve therapeutic outcomes. This requires a mechanism for direct delivery of tPA to the clot site, where it can act solely upon fibrin-bound plasminogen and minimize systemic plasminemia.

Several attempts have been made to achieve targeted delivery of fibrinolytic agents such as tPA to blood clot sites. For example, attempts have included creating complexes of tPA with antifibrin antibody. Formation of such complexes has been achieved by crosslinking tPA with antifibrin antibody via disulfide bridges and by engineering fusion proteins with antifibrin antibody conjugated to the N-terminus of tPA (see, e.g., U.S. Pat. Nos. 5,811,265 and 5,609,869 to Quertermous, et al.). However, these approaches were found to be unacceptable for clinical applications—the former because of low stability and heterogeneity of the crosslinked proteins, and the latter due to the inhibited fibrinolytic activity of tPA by the conjugated antibody.

In another design, tPA was modified by attachment of a positively charged cationic peptide and bound to a negatively charged heparin-antifibrin antibody conjugate via Coulombic interactions. The tPA-antibody complex remained inactive during delivery to the clot site. After delivery, tPA release was triggered by injection of protamine, which forms a highly stable complex with heparin. (Liang, et al., ATTEMPTS: a Heparin/Protamine-Based Delivery System for Enzyme Drugs, J. Controlled Release, 78, 67 (2002)). Although this system was effective in animal models, its thrombolytic activity was lower than that of free tPA.

Common problems associated with the use of these as well as other protein-protein conjugates as delivery vehicles include their potential immunogenicity, low stability in vivo, heterogeneity of functional properties due to crosslinking or chemical modification, and decreased fibrinolytic activity due to interaction with the antibody, genetic engineering, and/or chemical modifications.

What are needed in the art are methods and materials for targeted delivery of fibrinolytic agents such as tPA to targeted locations while maintaining the activity of the agents.

### SUMMARY

According to one embodiment, disclosed are conjugated particles. For example, a conjugated particle can include a carrier particle, a first polypeptide attached to the carrier particle and a second polypeptide attached to the carrier particle. The first polypeptide can be a fibrinolytic agent and the second polypeptide can target the conjugated particle to a component of a blood clot. For instance, the first polypeptide can catalyze the activation of plasminogen to plasmin, e.g., tissue plasminogen activator (tPA); and the second polypeptide can specifically bind to a component of a blood clot such as fibrin. In one particular embodiment, the second polypeptide can be an antifibrin antibody.

The conjugated particles can include additional materials bound thereto as well. For instance, a conjugated particle can include multiple different fibrinolytic agents and/or multiple different targeting agents. Other materials, such as blocking agents and the like can also be incorporated on the conjugated nanoparticles.

Carrier particles can be sized for a desired application. For instance, for in vivo applications it may be preferred to utilize nano-sized carrier particles, for instance, less than about 50 nm. In vitro applications may prefer larger carrier particles, for instance on a micrometer scale.

Polypeptides of the conjugated particles can be bound to the carrier particle either directly or indirectly. For instance, at least one of the first polypeptide and the second polypeptide can be indirectly bound to the carrier particle with a spacer, e.g., a polymeric spacer, there between. Additionally, polypeptides can be attached to a carrier particle with either covalent binding methodologies or non-covalently.

According to another embodiment, a conjugated particle can include a targeting agent, such as antifibrin antibody, and can also include a fibrinolytic agent that directly acts to degrade fibrin. For instance, a carrier particle can be bound to a substrate that, when activated, degrades fibrin. In one preferred embodiment, a conjugated particle can include a targeting polypeptide and plasminogen.

Also disclosed are methods of utilizing conjugated particles to break down fibrin enzymatically, and in one particular embodiment, fibrin contained in a blood clot. For instance, a conjugated particle can be bound to a component of a blood clot via the targeting agent of the particle. Upon association of the fibrinolytic agent carried by the particle with its substrate, e.g., plasminogen, the fibrinolytic agent can activate the substrate, and the activated substrate can then cleave the fibrin in the blood clot. According to one embodiment, the activation method can occur in solution, for instance, an in vivo method in which the substrate (e.g., plasminogen) is in serum near the particle.

Also disclosed are methods for forming conjugated particles. Methods can include binding to the carrier particle the two polypeptides. In one preferred embodiment, both polypeptides can be bound simultaneously, so as to simplify the formation process.

### BRIEF DESCRIPTION OF THE FIGURES

A full and enabling description of the presently disclosed subject matter, including the best mode thereof, to one of ordinary skill in the art, is set forth more particularly in the remainder of the specification, including reference to the accompanying Figures, in which:

FIG. 1 schematically illustrates one embodiment of a process for forming conjugated particles as disclosed herein;

FIG. 2 schematically illustrates another embodiment of a process for forming conjugated particles as disclosed herein;

FIG. 3 schematically illustrates another embodiment of a process for forming conjugated particles as disclosed herein;

FIG. 4 schematically illustrates a method for targeting disclosed conjugated particles to a fibrin-containing material;

FIG. 5 schematically illustrates a method for targeting multiple different conjugated particles to a fibrin-containing material;

FIG. 6 compares the activity in solution of conjugated particles including tethered tPA to that of free tPA;

FIG. 7 illustrates the decrease in fluorescence of solutions containing conjugated particles as disclosed herein upon binding of particles to a fibrin-containing clot;

FIGS. 8A and 8B compare the in vitro fibrinolytic activity of free tPA to that of several different conjugated nanoparticles as described herein that differ according to tethered tPA:antifibrin antibody (AF) ratio;

FIG. 9 illustrates in vitro fibrinolytic activity of conjugated particles as described herein compared with activity of free tPA, each point is an average of four kinetic experiments;

FIG. 10 illustrates atomic force microscopy (AFM) images of monodisperse polylactic acid (PLA) beads before (FIG. 10A) and after (FIG. 10B) covalent attachment of tPA and antifibrin antibody, also shown are cross sectional profiles (FIGS. 10C-10F) at several locations of the respective AFMs;

FIG. 11 compares tPA activity when covalently bound to polylactic acid (PLA) particles as compared to free tPA;

FIGS. 12A-12D; illustrate the kinetics of blood clot dissolution for free tPA as compared to tPA-PLA-AF conjugated particles formed as described herein

FIG. 13 illustrates the kinetics of fibrin clot dissolution utilizing tPA-PLA-AF conjugated particles formed as described herein; and

FIGS. 14A and 14B compare the stability over time of free tPA with that of tPA-PLA-AF conjugated particles formed as described herein.

### DETAILED DESCRIPTION OF REPRESENTATIVE EMBODIMENTS

### Definitions

The term “polypeptide” as utilized herein generally indicates a molecular chain of amino acids and does not refer to a specific length of the product. Thus, peptides, oligopeptides and proteins are included within the definition of polypeptide. This term is also intended to include polypeptides that have been subjected to post-expression modifications such as, for example, glycosylations, acetylations, phosphorylations and the like.

The term “protein” as utilized herein generally indicates a molecular chain of amino acids that is capable of interacting structurally, enzymatically or otherwise with other proteins, polypeptides or other organic or inorganic molecules.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made to the disclosed subject matter without departing from the scope or spirit of the disclosure. For instance, features illustrated or described as part of one embodiment, may be used with another embodiment to yield a still further embodiment.

Use of enzymes in both in vivo applications (e.g., drug delivery) as well as in vitro applications (e.g., assays for enzyme substrates) can offer multiple advantages because of their high catalytic activity and unique selectivity. One of the shortcomings of enzyme utilization, however, is the inability of enzymes to distinguish between a substrate present at a specifically targeted site versus that elsewhere in the system, for instance in solution. This limitation may make in vivo enzyme therapy unacceptable due to the risks of excessive or toxic side effects in normal tissues and may create unnecessary cost as well as false results during in vitro uses. The presently disclosed methods and materials, directed for use in delivering active fibrinolytic agents to targeted sites, can solve such problems.

Disclosed herein are conjugated particles that can be utilized in one embodiment to deliver one or more active fibrinolytic agents to a targeted site, and specifically, a site that includes a component common to blood clots, such as fibrin. More specifically, disclosed conjugated particles include a carrier particle and at least two polypeptides attached thereto, the first polypeptide being a fibrinolytic agent, for example an agent that is capable of catalyzing the activation of a substrate, the active form of which can enzymatically break down fibrin, and the second polypeptide being capable of targeting a conjugated particle to a component of a blood clot. Moreover, the target of the second polypeptide can be fibrin or can be a different component of a blood clot. For instance, the first polypeptide can be an active tissue plasminogen activator (tPA) protein that activates the substrate plasminogen to form the fibrinolytic enzyme plasmin and the second polypeptide can be an antifibrin antibody or a functional portion thereof that can specifically bind fibrin.

The use of a particle as a carrier of an active polypeptide can offer several important advantages over the use of purely protein-based systems. For instance, coupling of proteins to particles can be a relatively simple process to complete, and synthesis of conjugated particles can be accomplished within a matter of hours. In addition, a plurality of the same or different polypeptide molecules can be bound to a single particle (e.g., a 20 nm nanoparticle can host up to up to about 100 protein molecules). The total number of polypeptide molecules attached to each particle can be controlled as can the proportion of each different compound bound to a single particle. Thus, the activity level of a conjugated particle can likewise be controlled. Moreover, aggregation of particles can be minimized so as to achieve a uniform size distribution of protein-particle conjugates and conjugates can be much more stable as compared to, for example, crosslinked proteins.

In general, carrier particles can be on a micro- or nanoscale size, with a preferred size generally depending upon the desired application of the formed conjugated particles. For instance, when considered for use in vivo, a carrier particle can generally be preferred on a nanometer sized scale. For instance, the mean diameter of a nanoparticle for use in vivo can generally be less than about 500 nanometers, for instance less than about 200 nm, or less than about 100 nm, in one embodiment. In one particular embodiment, carrier nanoparticles can be less than about 50 nm in size, for instance about 20 nm in mean diameter.

When considered for use in vitro, for instance in an assay system, preferred particles can be larger, for instance carrier particle formed on a micrometer sized scale can be utilized. For example, in some embodiments, the mean diameter of a carrier particle can range from about 0.5 microns to about 1,000 microns, in some embodiments from about 0.5 microns to about 100 microns, and in some embodiments, from about 0.5 microns to about 10 microns. In one particular embodiment, a carrier particle can have a mean diameter of from about 1 to about 2 microns.

Generally, the particles can be substantially spherical in shape, although other shapes including, but not limited to, plates, rods, bars, irregular shapes, etc., are suitable for use. For instance, in one embodiment, elongated tubular shapes are encompassed. For example, carbon nanotubes can be utilized as carrier particles in one embodiment. As will be appreciated by those skilled in the art, the composition, shape, size, and/or density of the particles may widely vary. In general, a particle can be homogeneous across the particle material, for instance across the cross section of a spherical particle or across the wall of a tubular particle.

In one preferred embodiment, the carrier particles can be solid. As utilized herein, the term ‘solid’ generally refers to particles that do not contain a great deal of water within the material forming the particle and do not absorb a great deal of water in an aqueous environment such that the size and shape of a particle will not vary appreciably from a dry environment to an aqueous environment. For example, when held in an aqueous environment, a solid particle as described herein can contain an amount of water less than about 100% of the weight of the particle. For example, a solid particle can contain no more than about 75% of the weight of the particle as water when held in an aqueous environment. In other embodiments, the maximum water content of a solid particle can be less than about 20% of the weight of the particle, less than about 10%, less than about 5%, or less than about 1% of the weight of the particle.

Preferred materials for the carrier particles can depend upon the desired use. In general, any biocompatible particle can be utilized in forming disclosed conjugates. In one embodiment, polymeric particles can be utilized. For instance, polymeric particles including one or more natural or synthetic polymers such as, without limitation, polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, and the like, or an aldehyde, carboxyl, amino, hydroxyl, or hydrazide derivative thereof can be utilized. Carrier particles are not limited to polymeric materials, however, and other materials as may be utilized in forming carrier particles can include, without limitation, oxides such as silica oxide, titania oxide, zirconia oxide, and the like; metals, such as gold, silver, platinum, palladium, and the like; carbon; silicon; and so forth.

In one embodiment, carrier particles can be biodegradable. For instance, biodegradable particles formed from polylactic acid homopolymers and copolymers can be preferred for in vivo applications. For example, particles formed of poly(lactic-co-glycolic acid) (PLGA) copolymers and derivatives thereof can be utilized for in vivo applications.

Hydrogel-based carrier particles are also encompassed in the present disclosure. For instance, in one embodiment, a carrier particle can include a biocompatible hydrogel matrix. The term ‘hydrogel’ generally refers to a polymeric matrix that can be highly hydrated while maintaining structural stability, and can be contrasted with solid particles previously described. Suitable hydrogel matrices can include un-crosslinked and crosslinked hydrogels. In addition, crosslinked hydrogel carrier matrices can include hydrolyzable portions, such that the matrix can be degradable when utilized in an aqueous environment. For example, in one embodiment, the carrier matrix can include a cross-linked hydrogel including a hydrolyzable cross-linking agent, such as polylactic acid, and can be degradable in an aqueous, in vivo environment.

Hydrogel-based carrier particles can include natural polymers such as glycosaminoglycans, polysaccharides, and the like, as well as synthetic polymers, as are generally known in the art. A non-limiting list of hydrophilic polymeric materials that can be utilized in forming a hydrogel carrier particle can include dextran, hyaluronic acid, chitin, heparin, collagen, elastin, keratin, albumin, polymers and copolymers of lactic acid, glycolic acid, carboxymethyl cellulose, polyacrylates, polymethacrylates, epoxides, silicones, polyols such as polypropylene glycol, polyvinyl alcohol and polyethylene glycol and their derivatives, alginates such as sodium alginate or crosslinked alginate gum, polycaprolactone, polyanhydride, pectin, gelatin, crosslinked proteins peptides and polysaccharides, and the like.

A carrier particle can be conjugated with at least two polypeptides, one of which being a functional polypeptide that is a fibrinolytic agent, and the other being a targeting polypeptide that can specifically bind a conjugated particle to a component of a blood clot. In one preferred embodiment, disclosed particles can be conjugated with primarily or exclusively surface immobilized polypeptides rather than be formed as polymer-protein conjugates (e.g., hydrogel-protein conjugates) in which some or all of the functional and/or targeting polypeptides can be buried inside the polymer globule, as this latter design can decrease the efficiency of the system. In addition, particles that are conjugated with desired polypeptides following formation such that the polypeptides are primarily or exclusively surface immobilized can generally be characterized by more controllable size, less tendency to aggregation, and higher diffusion rates as compared to many hydrogel matrix/polypeptide systems that can be formed in a single-step process.

This is not a requirement of disclosed particles, however. For instance, in other embodiments, it may be preferred to include a functional polypeptide throughout the matrix of a degradable hydrogel carrier particle, and include the targeting polypeptides as primarily surface immobilized materials.

A polypeptide can be conjugated to a carrier particle via either nonspecific or specific interaction. For instance, in one embodiment, a polypeptide can be conjugated to a carrier particle via nonspecific charge-charger interaction between the two. According to another embodiment, a polypeptide can be conjugated to a carrier particle via specific noncovalent or covalent bond formation. Preferred attachment methods can generally depend upon the desired application of the formed conjugates. For instance, in those embodiments in which a system is designed to function in vivo, e.g., in the blood stream, a conjugated particle can be expected to encounter multiple collisions with materials, such as plasma proteins. Accordingly, covalent binding can be preferred in such an embodiment, to better ensure that functional and targeting polypeptides will not be dislodged or replaced by more abundant proteins encountered in the blood stream.

In one embodiment, a carrier particle can include surface reactive groups that can facilitate conjugation of the particle with a functional and/or targeting polypeptide. Surface reactive groups can include, without limitation, aldehyde, carboxyl, amino, hydroxyl, thiol, ester, bromoacetyl, iodoacetyl, epoxy and other reactive or linking functional groups, as well as residual free radicals and radical cations, through which a polypeptide coupling reaction can be accomplished either directly or indirectly. For instance, latex particles including chloromethyl surface groups as are available from IDC Latex of Eugene, Oreg.; and carboxy- or amine-modified particles as are available from Microspheres-Nanospheres, Inc. of Mahopac, N.Y., can be utilized.

In addition, although particles are often functionalized after synthesis, in certain cases, such as poly(thiophenol) particles, a particle can be capable of direct or indirect covalent linking with a protein without the need for further modification.

Carrier particles can be functionalized according to protocols as are known in the art. For instance, a carrier particle can be aminated at the surface via contact with an amine-containing compound such as 3-aminopropyltriethoxy silane. A surface functional group can also be incorporated as a functionalized co-monomer because the surface of the particle can contain a relatively high surface concentration of polar groups. For example, carboxylic groups can be activated on a particle surface using carbodiimide.

In another embodiment, a polypeptide can be indirectly bound to a particle through a spacer. For instance, a compound can be bound to the surface of a particle that can in turn bind a functional and/or targeting polypeptide. A spacer can be polymeric or monomeric, as desired. By way of example, succinimidyl 4-(N-maleimidomethyl)cyclohexane-1-carboxylate (SMCC), or alternatively Sulfo-SMCC, both of which are available from Pierce Biotechnology, Inc. of Rockford, Ill., and both of which include a succinimide group on one end and a maleimide group on the other, can be bound to a particle. For example, SMCC can be bound to an aminated substrate surface by way of the succinimide group, leaving the maleimide functional group available for directly or indirectly binding a polypeptide, for instance via a sulfhydryl group.

Surface activation and/or polypeptide coupling can occur in a buffer, such as phosphate-buffered saline (PBS) (e.g., pH of 7.2) or 2-(N-morpholino) ethane sulfonic acid (MES) (e.g., pH of 5.3).

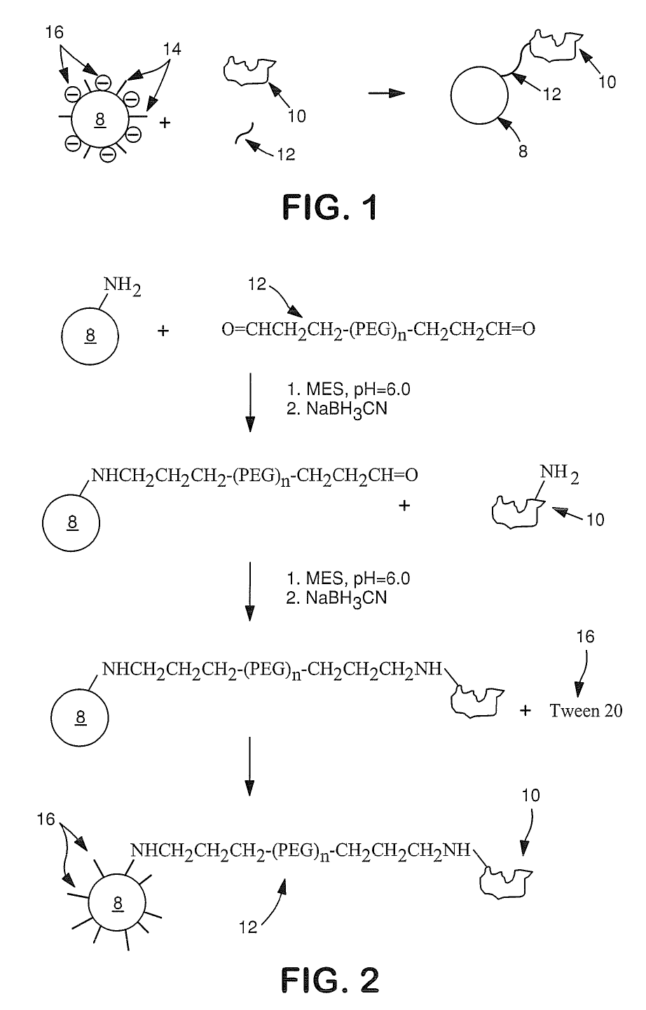

Referring to FIG. 1, one generalized schematic representation for forming disclosed conjugated particles is disclosed. According to this embodiment, a molecular spacer 12, for instance a hydrophilic spacer, can be utilized to tether a protein 10 to a carrier particle 8.

According to the disclosed subject matter, polypeptides that can be bound to a carrier particle can include a fibrinolytic agent and a targeting polypeptide. In one embodiment, a fibrinolytic agent of a conjugated particle can encompass an activator polypeptide that can act upon a substrate; the active form of which can degrade fibrin. For example, activator-type fibrinolytic agents can include plasminogen activators or analogs of plasminogen activators. Plasminogen activators as may be bound to conjugated particle can include, without limitation, tPA, urokinase, pro-urokinase, streptokinase, staphylokinase, and the like.

Activator fibrinolytic agents encompassed herein can include any activator polypeptide as would be known to one of skill in the art. For example, isolated tPA as may be bound to a carrier particle can include any functional tPA-containing polypeptide including native tPA, recombinant tPA, active variants of any tPA such as for instance glycosylated tPA, and so forth. By way of example, native human tPA available from Calbiochem of La Jolla, Calif., and U.S. Pat. No. 5,010,002 to Levinson et al. incorporated herein by reference discloses methods for producing recombinant human tPA, both of which can be utilized. Any active variant of tPA can alternatively be utilized in disclosed products and methods. For instance, tPA variants as described in U.S. Pat. Nos. 5,612,029 to Bennett, et al., and 5,407,819 to Yahara, et al., both of which are incorporated herein by reference, can be utilized as described herein. As other examples, U.S. Pat. No. 5,472,692 to Liu et al. and U.S. Pat. No. 7,074,401 to Gurewich, both incorporated herein by reference, describe pro-urokinase mutants; U.S. Pat. No. 7,163,817 to Sahni et al. incorporated herein by reference describes modified forms of streptokinase with enhanced fibrin selectivity; and U.S. Pat. No. 7,205,139 to Moore et al. incorporated herein by reference describes homologs of tPA. All of the foregoing can be used as described herein. Moreover, non-human tPA is also encompassed herein, for instance for in vitro applications and non-human in vivo applications.

According to one embodiment, a fibrinolytic agent component of disclosed conjugated particles can be a direct fibrinolytic agent that can directly act upon fibrin of a blood clot. For instance, direct fibrinolytic agents can include plasminogen, plasmin and metalloproteinases, or analogs of such. Metalloproteinases and analogs of metalloproteinases that can be bound to a conjugated particle can include, without limitation, matrix metalloproteinase-3 (MMP-3), fibrolase, alfimeprase, and the like.

A fibrinolytic agent that can directly act upon fibrin can be conjugated with a carrier particle in an active or an inactive form. For example, in one embodiment, discussed further below, an inactive fibrinolytic agent, such as plasminogen, can be conjugated to a carrier particle. Upon targeting of the conjugated particle to the desired location, the inactive fibrinolytic agent, e.g., plasminogen, can be activated to form the active form of the compound, which can then act upon fibrin at the targeted site.

In addition, it should be understood that a carrier particle is not limited to carrying only a single fibrinolytic agent. For example, a carrier particle can be conjugated with multiple activator fibrinolytic agents, e.g., multiple plasminogen activators, e.g., tPA and urokinase, or with multiple direct fibrinolytic agents, e.g., fibrolase, MMP-3, and plasminogen, as well as any combination thereof. In general, an activator fibrinolytic agent such as tPA will not be conjugated to the same particle as its substrate, e.g., plasminogen, so as to prevent activation of the inactive protein prior to targeting completion.

In order to specifically target a particle for delivery to a blood clot, one or more specific binding members for components of the blood clot can also be conjugated to a particle. For example, in one embodiment, a specific binding member for fibrin, e.g., an antifibrin antibody, can be bound to a carrier particle. Specific binding members for fibrin as may be attached to a carrier particle can include complete antifibrin antibodies or functional fragments thereof such as the F_{ab}, F_{(ab′)2} fragments, single chain antibodies (Fᵥ) and the like. For instance, U.S. Pat. Nos. 5,011,686 and 5,443,827, both to Haber et al. and both incorporated herein by reference, disclose monoclonal antibodies specific to fibrin, which can be used as described herein. Moreover, other types of specific binding members can be used to target fibrin. For example, U.S. Pat. No. 5,288,490 to Budzynski et al. incorporated herein by reference describes fibrin fragments that bind to fibrin polymers found in blood clots, and U.S. Pat. No. 6,984,373 to Wescott et al. incorporated herein by reference discloses synthetic polypeptides with high specific affinity for the form of polymerized fibrin found in blood clots. All of the foregoing can be used as described herein.

Targeting polypeptides of disclosed conjugated particles are not limited to fibrin targeting polypeptides. Specifically, a conjugated particle can include one or more targeting polypeptides that can specifically bind any component that can be found in a blood clot. For example, a targeting polypeptide can specifically target blood clot components such as fibronectin, activated platelets, fibrin bound α2-antiplasmin, tissue factor, gpIIb/IIIa, tissue factor/VIIA complex activated clotting factor Xa, activated clotting factor IXa, fibrin condensation product d-dimer, and so forth. For example, U.S. Pat. Nos. 5,582,862; 5,831,031; and 6,114,506, all to Reed, et al. and incorporated herein by reference, describe antibodies and methods for forming the antibodies directed to α2-antiplasmin crosslinked to fibrin (α2AP-FX) which does not inhibit plasma α2-antiplasmin (α2AP) and may be utilized as targeting polypeptides as described herein. By way of additional examples, U.S. Pat. No. 5,633,986 to Tait et al. and incorporated herein by reference describes annexins that have high affinity for activated platelets, and U.S. Pat. No. 5,951,981 to Markland et al and incorporated herein by reference describes peptide ligands that specifically bind to gpIIb/IIIa.

A targeting polypeptide can include a specific binding member of a blood clot component. The term ‘specific binding member’ generally refers to a member of a specific binding pair, i.e., two different molecules where one of the molecules chemically and/or physically binds to the second molecule. For instance, immunoreactive specific binding members can include antigens, haptens, aptamers, antibodies, and complexes thereof, including those formed by recombinant DNA methods or peptide synthesis. An antibody can be a monoclonal or polyclonal antibody, a recombinant protein or a mixture(s) or fragment(s) thereof, as well as a mixture of an antibody and other specific binding members. The details of the preparation of such antibodies and their suitability for use as specific binding members are well known to those skilled in the art.

A specific binding member can be prepared, for example, using any variation of phage display protocol or the like as are generally known in the art, to provide a polypeptide for targeting and binding a particle to fibrin. A targeting polypeptide, e.g., an antifibrin antibody or functional portion thereof can be monoclonal or polyclonal, as desired. For example, monoclonal anti-human fibrinogen antibody (clone MFB-HB) available from Accurate Chemical & Scientific Corp. of Westbury, N.Y., or mouse monoclonal fibrin antibody available from GeneTex of San Antonio, Tex., can be utilized. In one embodiment, antifibrin monoclonal antibody (NIB 1H10, NIB 12B3, NIB 5F3, etc.) can be produced and purified by conventional methods and bound to a carrier particle.

Moreover, a polypeptide can include additional materials bound thereto. For instance, a polypeptide can include a detectable label bound thereto, such as a fluorescent label as may be detected in an assay protocol. Detectably labeled polypeptides and methods for forming labeled polypeptides are generally known in the art. For instance, FITC-labeled tPA available from American Diagnostica Inc. can be bound to a carrier particle. Methods for forming labeled polypeptides as are generally known in the art can optionally be utilized.

In addition, it should be understood that a conjugated particle can include multiple different targeting polypeptides. For example, a conjugated particle can include two or more different targeting polypeptides, each of which targets the same component of a blood clot; different targeting polypeptides, each of which targets different components of a blood clot; or combinations thereof.

Referring again to FIG. 1, a conjugated particle can include a spacer 12 linking a polypeptide 10 to a carrier particle 8. For instance, a polymeric spacer 12 can be bound to a surface reactive group 14 at a first end, and a functional or targeting protein 10 can bind at the other end of the polymeric spacer 12. Utilization of a spacer 12 as illustrated in FIG. 1 can prevent interaction of a covalently bound protein 10 with the particle surface and thus prevent structural changes of the protein 10 that can lead to partial or complete loss of functionality. A polymeric spacer 12 can include long (e.g., M, between about 2,000 and about 20,000 Da) hydrophilic polymers. Exemplary hydrophilic polymeric spacers can include, without limitation, poly or oligo(ethylene glycol), polyvinyl alcohol, polysaccharides, and the like. In the illustrated embodiment, a carrier particle 8 can be stabilized through the addition of charged groups 16 at the surface of the particle 8, as shown.

FIG. 2 schematically illustrates one method of attachment of a polypeptide 10 to an aminated carrier particle 8 through a spacer 12. For example, a bifunctional PEG spacer 12, e.g., a bispropionaldehyde-PEG spacer 12 can be covalently attached to the carrier particle 8 via reaction between an aldehyde group of the spacer 12 with a surface amine group of the particle 8. This reaction results in particles 8 including a propion-aldehyde-terminated spacer 12. A polypeptide 10 can then be attached to the spacer 12 according to a simple process including mixing of a protein solution with an aqueous suspension of particles so as to bind the protein 10 at the aldehyde groups of the spacer 12 via an amine of the protein 10.

Specifics of protein binding to a spacer can be controlled according to disclosed methods. For instance, a propionyl-aldehyde terminated spacer can selectively react with the N-terminal amino groups of polypeptides at about pH 5. However, attachment of a polypeptide via the N-terminal amino group may not be preferred in some embodiments. For instance, attachment of tPA via its N-terminus may interfere with its activity, as indicated in previous studies on construction of fusion proteins with an antibody conjugated to the N-terminus of tPA. In contrast, attachment of tPA to a particle via random amine groups throughout the polypeptide appears to interfere little or not at all with the fibrinolytic activity of the protein. Thus, in one preferred embodiment as illustrated in FIG. 2, propionyl-aldehyde based coupling of a carrier particle to an active protein can be carried out at about pH 6 (e.g., using an MES buffer), so as to provide binding that does not discriminate between the N-terminal and internal amine groups, e.g., at lysine. As a result, improved activity of the protein can be obtained. According to one embodiment, the propionyl-aldehyde group can be reduced, e.g., with sodium cyanoborohydride, to bind random amino groups of the enzymes

At the final stage of the conjugation illustrated in FIG. 2, the conjugated particle can be blocked, for instance, with a surfactant, such as Tween® 20, Pluronic®, ethanolamine, or dextrane that can be adsorbed on the particle to block any hydrophobic surface exposed to the solution as well as to displace noncovalently bound proteins. Low concentrations of such materials generally do not interfere with the activity of water soluble enzymes such as those disclosed herein. The presence of a surfactant at the surface of the conjugated particle can reduce undesirable protein-particle interactions and prevent particle aggregation. It can also prevent the nonselective “fouling” of the surface of the particles with materials encountered during use, such as plasma proteins, that could deactivate a delivery system.

According to another embodiment, a conjugated particle can include polylactic acid-based carrier particles, for instance, carboxyl-modified polylactic acid (PLA) nanoparticles can be utilized as carrier particles. According to one such embodiment, a spacer including the same or different functional groups on either end of the spacer, for example an NH₂-PEG-COOH spacer, can be bound to the carboxyl functionality at the surface of a particle through the terminal amine group using carbodiimide chemistry according to known methodology. A functional or targeting protein can then be coupled to the carboxyl group of the spacer using carbodiimide chemistry. The surface of the nanoparticle can then be blocked with a suitable agent (e.g., Tween® 20, Pluronic®, dextran, and the like), as described above. Blocking agents can be adsorbed, absorbed, or bonded to the surface of a particle, according to known methodology.

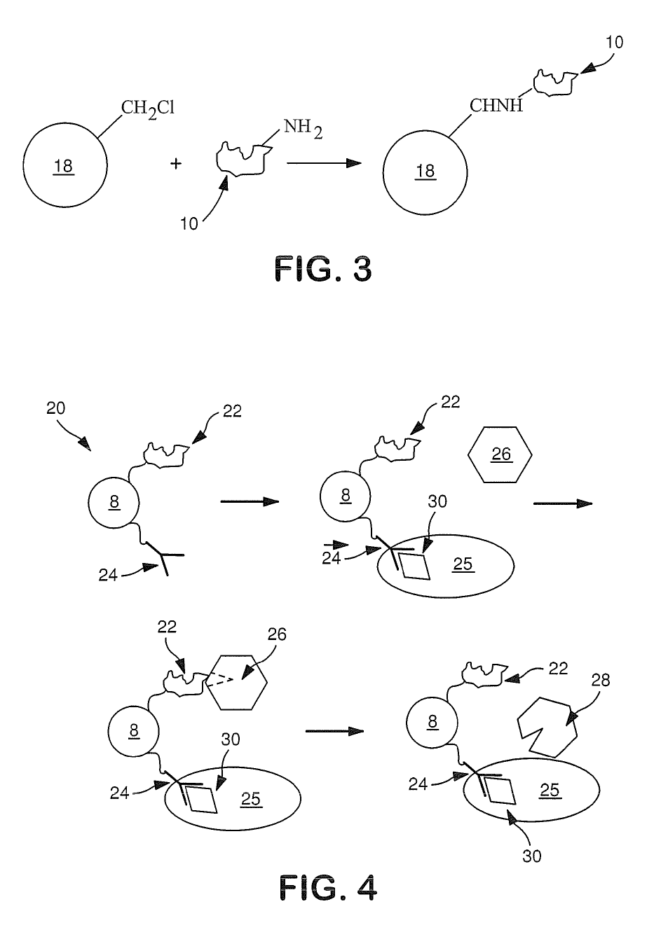

While the indirect tethering of a protein to a carrier particle via a long polymeric spacer can be preferred in some embodiments, it should be understood that utilization of a long molecular spacer between the carrier particle and a bound protein is not a requirement of the disclosed subject matter, and in other embodiments, active proteins can be bound to carrier particles without the addition of a long spacer between the two. For instance, FIG. 3 schematically illustrates another method for forming conjugated particles as disclosed herein. In this embodiment, a protein 10 can be bound to a chloromethylated carrier particle 18 according to a nucleophilic substitution reaction directly between protein amine groups of the protein 10 and alkyl chloride surface groups of the particle 18.

According to yet another embodiment, soluble carbodiimide (EDC) and glutaraldehyde chemistry can be used to achieve covalent binding of protein amine groups to carboxylated and aminated particles, respectively.

Polypeptides can also be bound to a carrier molecule through a streptavidin/biotin binding protocol. For example, a streptavidin monolayer can be bound to a particle surface followed by controllable attachment of desired amounts of biotinylated proteins. The presence of a streptavidin monolayer at the surface of a carrier particle, similar to other blocking agents, can also eliminate potential problems associated with interaction of functional proteins with a particle surface.

By way of example, streptavidin can be coated on a particle surface via functionalization added to the streptavidin that allows immobilization on the surface. For example, streptavidin may be immobilized on a gold particle through incorporation of thiol groups into the streptavidin structure. In another embodiment, streptavidin may be modified to contain any one of a number of silane functional groups that may allow streptavidin immobilization on a silicon-based particle. In yet another embodiment, biotin may first be adsorbed onto a particle surface, such as through a reaction between amine groups on an aminated particle surface and a modified biotin, for example a succinimide-modified biotin, and streptavidin may then be immobilized onto the surface via the adsorbed biotin. These are merely exemplary techniques, however, and any suitable coating method known in the art may be utilized for the purpose of immobilizing streptavidin on a particle surface. A biotinylated protein as can be formed according to standard methodology as is known in the art, can then be bound to the streptavidin-coated particle via the streptavidin/biotin binding.

When considering a binding protocol that utilizes amine groups of a polypeptide, as polypeptide molecules normally have more than one amine group, a single polypeptide molecule can potentially bind to more than one carrier particle. This may result in the formation of dimers and larger aggregates of carrier particles. While formation of large aggregates may be preferred in some embodiments, for instance in some in vitro assay applications, in other applications, it can be preferable to minimize aggregation. Accordingly, in some embodiments, a formation protocol including lower particle concentration and/or a higher concentration of surfactant, as well as variation in surfactants, can be utilized during a formation process in order to minimize aggregation of conjugated particles.

Conjugated particles can also be formed so as to include a predetermined ratio of functional polypeptides to targeting polypeptides. For instance, in one embodiment, binding protocols for both types of polypeptides can be the same, e.g., a carrier particle or spacer groups bound thereto can include carboxyl functional groups and both types of polypeptides can bind via amine groups of the polypeptides. According to this embodiment, polypeptides can bind via incubation of a solution including both polypeptides at a predetermined ratio with the carboxyl functionalized carrier particles. Depending upon the ratio of the different polypeptides in the solution and the binding efficiency of the different polypeptides to the carrier particle, a conjugated particle can be formed including a predetermined ratio of functional to targeting polypeptide. Additional details of binding efficiencies and activity levels of formed conjugated particles are provided below in the example section. Moreover, development of any specific system is well within the abilities of one of ordinary skill in the art.

Conjugated particles can be utilized to bind one or more fibrinolytic agents at a blood clot or a material comprising one or more components of a blood clot. For instance, as illustrated in FIG. 4, a conjugated particle 20 including a carrier particle 8, a fibrinolytic agent 22 and a targeting agent 24 can specifically bind a target 30, e.g., fibrin, via the targeting agent 24. In this particular embodiment, the fibrinolytic agent is an activating agent such as tPA. As the substrate 26 for the activating agent 22 approaches the location of the bound conjugated particle 20, the activating agent 22 of the conjugated particle 20 can activate the substrate 26, forming the fibrinolytic protein 28 in close proximity to the fibrin 30, such that the activated fibrinolytic protein 28 can cleave the fibrin 30.

Disclosed conjugated particles can be utilized in both in vitro and in vivo applications, as desired. For example, disclosed protein-particle conjugates can be utilized as therapeutic agents for treatment of acute myocardial infarction, ischemic stroke, deep vein thrombosis, and the like. For example, disclosed conjugates can deliver and hold an activator fibrinolytic agent at a blood clot in vivo and thus target substrate activation in the area of the clot. In one specific embodiment, disclosed methods can hold tPA in the area of a blood clot so as to target plasminogen activation in that area. As such, treatment methods utilizing the conjugated particles can utilize lower concentrations of an activator fibrinolytic agent such as tPA in a treatment protocol and prevent generation of excessive systemic activated substrate, e.g., plasmin. In addition, and as described in more detail below, the activity level of disclosed conjugated particles has been found to be lower than that of free activator fibrinolytic agents when the particles are in solution, i.e., not bound to fibrin. Upon binding to fibrin, however, the activity levels of the agents bound to the conjugated particles can approach or even exceed that of the free agent. Hence, protocols incorporating disclosed conjugated particles can be less likely to increase serum levels of the activated substrate as compared to similar, unbound agents, and as such can prevent conditions such as systemic plasminemia.

Moreover, and while not wishing to be held to any particular theory, it is believed that the stability of fibrinolytic agents bound to particles can exceed that of free fibrinolytic agents. Specifically, other types of proteins conjugated to polymers have been shown to possess prolonged plasma elimination half-life (5- to 500-fold increases in elimination half-life have been reported) and reduced proteolytic degradation rate. Similar stability is believed to occur in the disclosed conjugated particles.

Disclosed conjugated particles can also beneficially be utilized for in vitro applications. For example, disclosed conjugated particles can beneficially be utilized in determining the presence or concentration of a substrate such as plasminogen in samples. In one particular embodiment, a conjugated particle can be delivered and held at a surface, and can be utilized to determine the presence or concentration of plasminogen in samples including extremely low concentrations of a targeted plasminogen substrate. For instance, a clot lysis assay can be utilized including solidified fibrin-containing agarose gel plates. According to this embodiment, solutions containing disclosed conjugated particles can be added to microplate wells containing the solidified fibrin agarose gel and incubated to allow the antifibrin antibody (AF) of the conjugates to bind the fibrin. Upon incubation with a solution including plasminogen, the plasminogen in the sample can be activated to plasmin by the tPA bound to the particles and subsequent fibrinolysis can be quantified by comparing the size of the lysed zone around the sample wells with standards, for instance standards prepared by addition of known concentrations of free plasminogen in a similar system. As a high percentage of formed plasmin can be expected to act on the fibrin due to proximity of activation, an assay incorporating disclosed conjugated particles can be utilized to determine extremely low levels of plasminogen in a sample.

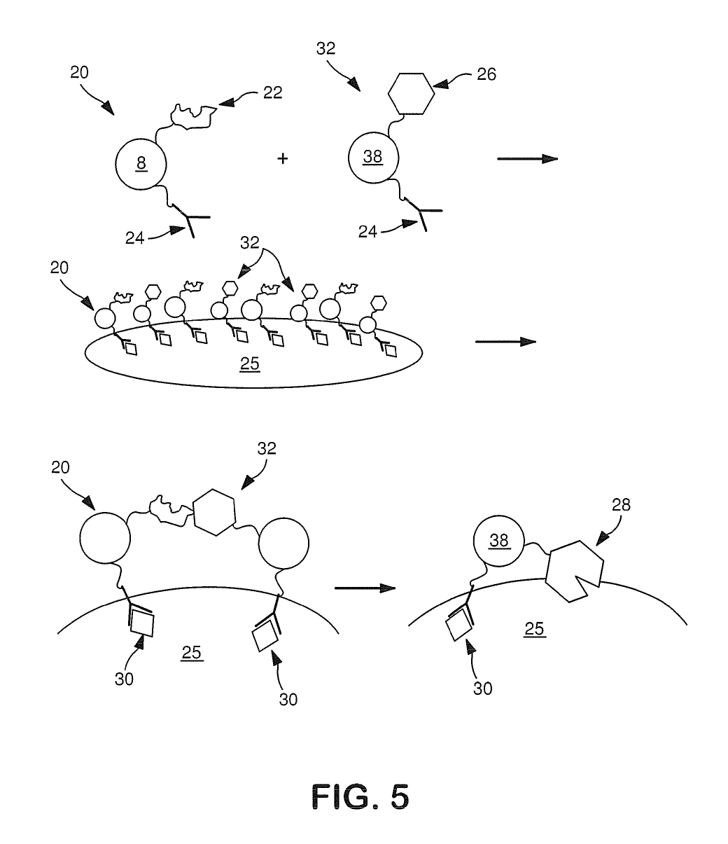

In another embodiment, illustrated in FIG. 5, conjugated particles as described herein can deliver both a substrate 26, such as plasminogen, and an activator for that substrate 22, such as tPA, to a blood clot 25. For instance, activator-AF conjugated particles 20 can be utilized in conjunction with substrate-AF conjugated particles 32 in order to bind both the substrate 26 and the substrate activator 22 to a blood clot 25. As illustrated in FIG. 5, substrate-containing conjugated particles 32 can be introduced into the system and directly delivered to a blood clot 25 via a specific binder 24, for the target 30, e.g., fibrin. In addition, activator-containing conjugated particles 20 can be delivered to the blood clot 25 in a similar fashion using the same or different specific binder 24. As targeted delivery can greatly concentrate the activator 22 near the particle-linked substrate 26, an even lower concentration of the activator 22 can be used to encourage cleavage of the fibrin. Upon interaction of the activator 22 with the substrate 26, the active form of the substrate 28 can be formed at the blood clot 25 and can then act upon the fibrin in the blood clot 25.

Reference now will be made to various embodiments, one or more examples of which are set forth below.

### Example 1

FITC-labeled tPA (70 kDa, American Diagnostica, Cat. #171) and Rhodamine-labeled antifibrin antibodies (Antifibrin MAb, clone 59D8, 150 kDa, GeneTex, Cat. #GTX19079) were simultaneously attached to 20 nm chloromethylated (CM) polystyrene latex beads (IDC Latex) via interaction of protein amine groups with the surface CH₂Cl— groups of the beads. The conjugation of tPA and AF to the CM nanoparticles was based upon the process of nucleophilic substitution with alkyl chlorides, as schematically illustrated in FIG. 3. Two samples with different initial concentrations of FITC-tPA and Rhodamine-AF corresponding to 25 tPA and 100 AF molecules per nanoparticle (sample 25tPA100AF), 50 tPA and 100 AF molecules per nanoparticle (sample 50tPA100AF), 100 tPA and 100 AF molecules per nanoparticle (sample 100tPA100AF), 100 tPA and 50 AF molecules per nanoparticle (sample 100tPA50AF) and 100 tPA and 25 AF molecules per nanoparticle (sample 100tPA25AF), respectively, were prepared. After overnight incubation in 2 mM MES buffer, 0.05% Tween 20 was added to remove any physically adsorbed proteins. Following which the protein-nanoparticle conjugates were separated from the unbound proteins by centrifugation.

The amount of each of the attached proteins per nanoparticle was determined by analysis of total protein content in the conjugates using bicinchonic acid (BCA) assay and fluorimetric determination of the AF antibody. Results are shown in Table 1, below. As can be seen, tPA binds much better than AF in spite of the fact that their isoelectric points are similar (probably due to smaller molecular weight).

TABLE 1

| | Sample (molecules/nanoparticle | | | | |
|---|---|---|---|---|---|
| | 25 tPA | 50 tPA | 100 tPA | 100 tPA | 100 tPA |
| | 100 AF | 100 AF | 100 AF | 50 AF | 25 AF |
| tPA:antifibrin | 25:100 | 50:100 | 100:100 | 100:50 | 100:25 |
| added | | | | | |
| tPA:antifibrin | 17:10 | 19:11 | 18:13 | 17:7 | 18:4 |
| covalently | | | | | |
| attached | | | | | |

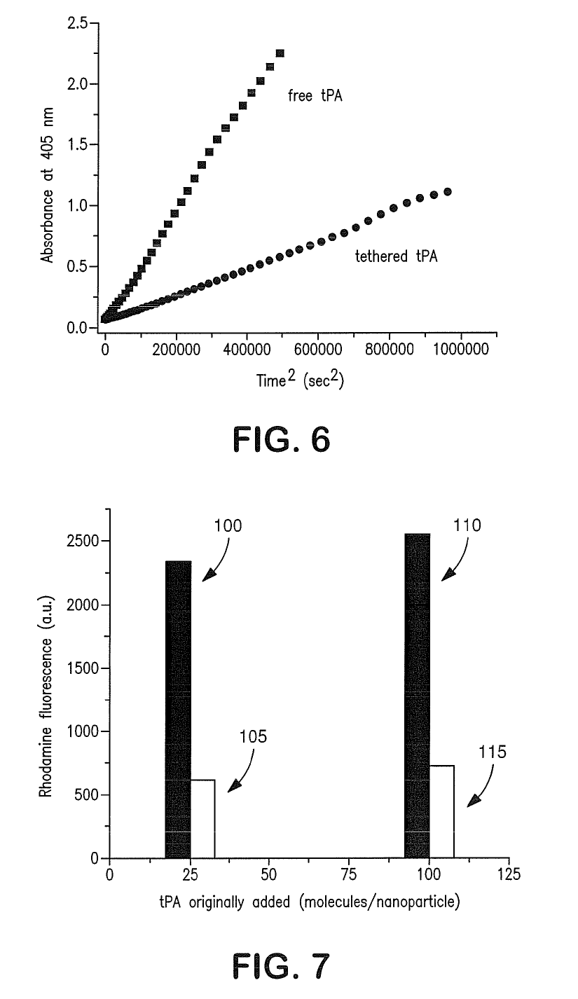

The activity of the tPA conjugates in the absence of fibrin clots was measured by monitoring the reaction of a soluble chromogenic substrate S-2251 with plasmin produced from tPA cleavage of plasminogen. In this assay the activity was calculated as the initial slope of the kinetic curve linearized in coordinates [absorbance]−[time]². Results for sample 100tPA100AF are shown in FIG. 6. As can be seen, the activity of conjugated tPA for both samples was about 30% of that of free tPA.

Fibrin clots were prepared in microplate wells by interaction of fibrinogen and thrombin. Targeting efficiency of the AF-tPA-nanoparticle conjugates to fibrin clots was assayed by comparing fluorescence intensities of initial suspension of AF-tPA nanoparticles (100, 110) with those of the supernatant obtained after 30-min incubation of the nanoparticle suspension with the fibrin clot (105, 115). It was found that for both 25tPA100AF (100, 105) and 100tPA25AF (110, 115) samples fluorescence dropped at least 60% after incubation with the clot, suggesting that approximately two-thirds of AF-tPA nanoparticles bound to the clot (FIG. 7).

In vitro fibrinolysis assay for samples 25tPA100AF and 100tPA25AF as well as for conjugated nanoparticles formed according to the same method with differing concentrations of AF and tPA, and also with free tPA, was performed using the reference method reported by Longstaff and Whitton (J. Thrombosis and Haemostasis, 2, 1416 (2004)). Activity of tPA was studied as a function of its concentration. Net tPA concentrations were identical for all compared systems and consisted of 2, 20, 200, and 2000 ng/mL.

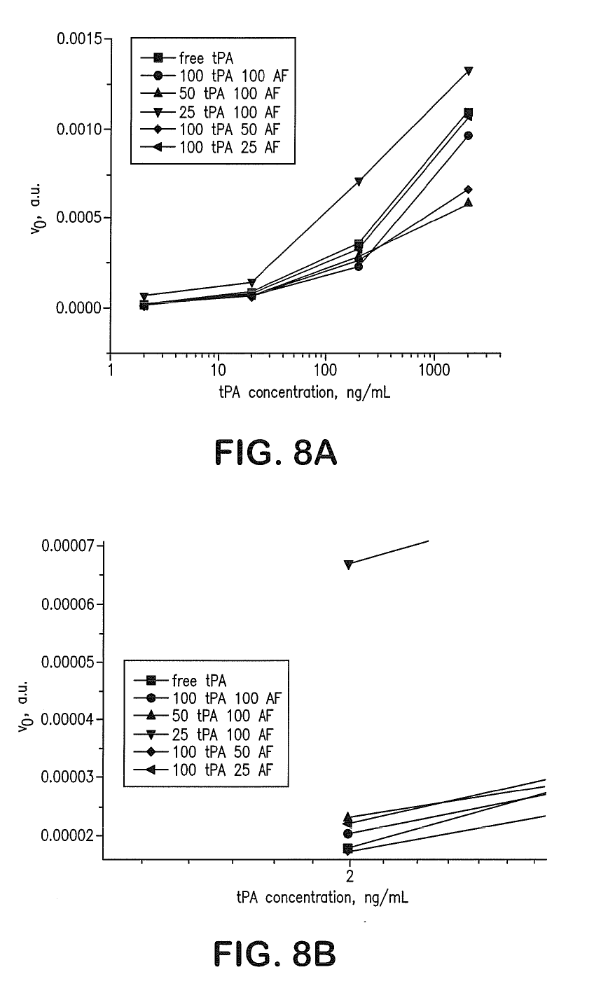

Results are shown below in Table 2 and in FIGS. 8A and 8B. For 25tPA100AF, the activity of AF-tPA-nanoparticles exceeded that of free tPA at all concentrations; this was likely due to more effective targeting to fibrin clots. At low tPA concentrations, the effect of the targeting was expected to be more pronounced. At tPA concentration of 2 ng/mL, sample 25tPA100AF was approximately four times more potent than free tPA, while at 2000 ng/mL rates of fibrinolysis for all samples were not significantly different. Fibrinolytic activity of 100tPA25AF was lower than that of 25tPA100AF, believed to be due to lower antibody and tPA content per nanoparticle. Notably, the activity of this sample was still similar to that of free tPA in spite of the fact that activity in the absence of fibrin clots was more than three times lower that that of free tPA. This indicates that effective targeting to fibrin clots increases the rate of fibrinolysis. At present it is not known why fibrinolytic activity of tPA-AF-nanoparticles exceeds that of free tPA, while activity in the absence of fibrin is lower than that of free tPA. It is hypothesized that antibody-directed binding to the clot brings tPA to the close proximity to and results in its enhanced activation by fibrin. Free tPA, on the other hand, has lower binding constant to fibrin, and, especially at low concentrations, significant fraction of free tPA remains unbound to and therefore is not activated by fibrin.

TABLE 2

| tPA, ng/mL | 2000 ng/mL | 200 ng/mL | 20 ng/mL | 2 ng/mL |
|---|---|---|---|---|
| Free tPA | 1.09 · 10⁻³ | 3.6 · 10⁻⁴ | 8.9 · 10⁻⁵ | 1.8 · 10⁻⁵ |
| 25tPA100AF | 1.3 · 10⁻³ | 7.1 · 10⁻⁴ | 1.4 · 10⁻⁴ | 6.7 · 10⁻⁵ |
| 100tPA25AF | 1.07 · 10⁻³ | 3.26 · 10⁻⁴ | 7.94 · 10⁻⁵ | 2.2 · 10⁻⁵ |

### Example 2

Negatively charged 40 nm polystyrene latex nanoparticles with chloromethyl surface groups were purchased from IDC Latex (Eugene, Oreg.). Human lyse-plasminogen (83 kDa) was obtained from Haematologic Technologies (Essex Junction, Vt.) in a buffer of 50% Glycerol, 20 mM HEPES, and 150 mM NaCl, and stored at −80° C. in aliquots of 50 μL each with a concentration of 1 mg/mL. The lyophilized powder of monoclonal anti-human fibrinogen antibody, clone MFB-HB (AF) was purchased from Accurate Chemical & Scientific Corporation (Westbury, N.Y.) and was stored in darkness at 4-8° C. Before synthesis, the antibody was reconstituted to obtain protein concentration of 1 mg/ml. Fluorescently labeled antibody was prepared using rhodamine succinimydyl ester labeling kit (Invitrogen, Carlsbad, Calif.). Fluorescently labeled antibody was purified by dialysis with a 5,000 kDa membrane. Calbiochem (La Jolla. Calif.) supplied both the human tissue plasminogen activator (65 kDa), and the citrate-free, thrombin from human plasma (37 kDa). The human tissue plasminogen activator (tPA) was reconstituted in HPLC-grade water and aliquots of 100 μL, each with a concentration of 1 mg/mL, were stored at −20° C. Thrombin was also reconstituted in HPLC-grade water and stored in aliquots containing 0.96 U at −80° C.

The conjugation of proteins to chloromethylated nanoparticles was based on the process of nucleophilic substitution with alkyl chlorides (FIG. 3). Before conjugation, tPA and AF antibody were dialyzed in 2 mM MES buffer. Suspension of chloromethylated nanoparticles provided by the supplier was diluted five-fold (1.8×10¹⁴ particles per mL) and dialyzed in 2 mM MES buffer. Sample volumes were measured before and after the dialysis, and change of the sample volume, if observed, was taken into account during the further calculations. For conjugation, dialyzed suspension of nanoparticles was added to solutions containing different concentrations of tPA and AF antibody. Three initial mixtures with compositions corresponding to 100:100, 50:100, and 100:50 tPA and AF molecules per nanoparticle, respectively, were prepared (samples 100-100, 50-100, and 100-50, respectively). To verify reproducibility, two samples were independently prepared for each of the compositions.

After allowing for overnight attachment of tPA and AF to the nanoparticles, 0.1 wt. % of Tween 20 was added to the samples to serve as a stabilizer and remove any physically adsorbed proteins. After 15 min incubation with Tween20, 50 μL of the colloidal suspension was removed to represent the initial amount of proteins in the solution. Following, the remaining volume of each sample was centrifuged at 30,000 g for 2 h, leaving free (unattached) proteins in the liquid supernatant and tethered (attached) proteins in the pellet. All of the supernatant was removed, and the pellet was resuspended in the amount of 20 mM MES buffer equivalent to the amount of the removed supernatant. Resuspended protein-nanoparticle conjugates were then vortexed and sonicated for 5 min yielding stable colloidal suspensions. Thus prepared suspensions were stable for weeks without sedimentation.

Rhodamine-labeled antibody was used to determine binding yield for the AF. 50 μL of each of the three solutions prepared in the previous step (initial suspension, supernatant, and the resuspended pellet) were placed into a Costar transparent 96-Microwell plate. Fluorescence of the samples was measured at the excitation/emission wavelengths of 590/645 nm using Synergy HT microplate reader (Bio-Tek). The amount of tethered AF was determined by comparison of the fluorescence intensities of the resuspended tethered tPA-AF nanoparticles to the fluorescence intensity of the supernatant and initial suspension. The difference between the fluorescence intensity of the initial suspension and the sum of the intensities of the corresponding supernatant and the resuspended pellet never exceeded 10%.

BCA assay (Pierce) was then used to determine total protein amount (tPA+AF) in the samples. The amount of tethered AF determined from the fluorescence assay was subtracted from the amount of total tethered protein determined from the BCA assay in order to yield the amount of tethered tPA when conjugated with AF (Table 3, below). Attempts to fluorescently label tPA by a second dye resulted in total loss of enzymatic activity.

TABLE 3

| Sample | NP/ml | Molecules tPA/NP | Molecules AF/NP |
|---|---|---|---|
| 100-100 | 1.8 × 10¹⁴ | 34.32 | 11.11 |
| 100-50 | 1.8 × 10¹⁴ | 46.38 | 6.6 |
| 50-100 | 1.8 × 10¹⁴ | 19.16 | 12.13 |

Each of the syntheses was reproduced in duplicate, as shown in Table 3. As can be seen from Table 3, yield of covalent binding for both tPA and AF is similar for different concentrations used, and consists of about 40% and about 10% for tPA and AF, respectively. These binding yields show good reproducibility for independent experiments.

Fibrin clots were prepared in microplate wells by interaction of fibrinogen and thrombin. First, 75 IA of fibrinogen (5 mg/mL) was added to a well, followed by 64 of plasminogen (1 mg/mL) and finally 75 μL of thrombin (0.0375 U). All protein solutions were prepared in 40 mM Tris-HCl buffer (pH=7.4) containing 0.2 wt. % of Tween20 and 0.5% wt. NaCl. The latter was added to ensure optical transparency of the clots. The fibrinogen and thrombin were incubated for 30 minutes, forming transparent gels comprised of a fibrin mesh.

Targeting of nanoparticles to fibrin clot was assayed by comparing fluorescence intensities of initial suspension of tPA-AF nanoparticles with that of the supernatant obtained after 30 and 90 min of incubation of nanoparticle suspension with the fibrin clot.

In vitro fibrinolysis assay for protein-nanoparticle conjugates, as well as for free tPA, was performed using the reference method reported by Longstaff and Whitton. Transparent fibrin clots were formed in microplate wells as described above. A mixture of tPA-AF nanoparticles conjugates or free tPA at 10, 100, 200, and 500 ng/mL and chromogenic substrate (S-2251) in 40 mM Tris-HCl buffer (pH=7.4) was added to the clot followed by mineral oil to prevent evaporation and clot shrinkage. Fibrinolysis occurred as tPA penetrated the clot and cleaved plasminogen present in the clot. The plasmin generated digested fibrin and also hydrolyzed the chromogenic substrate, S-2251, present as a reporter in the solution phase. The generation of p-nitroaniline was monitored by following the increase in optical density at 405 nm for 4 h. Each kinetic experiment was performed in duplicate.

Targeting efficiency of AF-tPA-nanoparticle conjugates to fibrin clots was assayed by comparing fluorescence intensities of initial suspension of AF-tPA nanoparticles with those of the supernatant obtained after 30 min incubation of the nanoparticle suspension with the fibrin clot. Analysis of the loss of fluorescence intensity showed that 65 to 70% of nanoparticles were bound to the clot for all samples. Experiments with longer incubation times (1.5 h) demonstrated that nanoparticle-antibody conjugates showed better attachment to fibrin clots than free antibody. It was speculated that stronger binding to the clots was observed due to the presence of multiple binding moieties on each nanoparticle (each conjugate contains 6-13 antibody molecules).

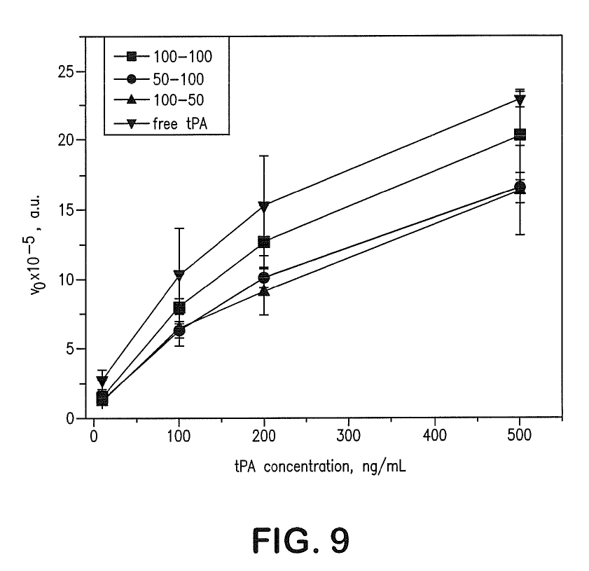

In vitro fibrinolysis activity of tPA conjugated to nanoparticles was studied as a function of concentration for two sets of independently prepared samples. Net tPA concentrations were identical for all compared systems and consisted of 10, 100, 200, and 500 ng/mL (physiological concentration of tPA is about 10 ng/mL). FIG. 9 shows results of the fibrinolysis assay. Activity for sample 100-100 was statistically not different from that of free tPA, while activities for samples 50-100 and 100-50 were slightly lower and consisted of 60-70% of that for free tPA (Table 4, below).

TABLE 4

| tPA, ng/mL | 500 ng/mL | 200 ng/mL | 100 ng/mL | 10 ng/mL |
|---|---|---|---|---|
| Free tPA | 23 0 ± 6 × 10⁻⁵ | 15.2 ± 3.6 × 10⁻⁵ | 10.3 ± 3.4 × 10⁻⁵ | 2.7 ± 0.8 × 10⁻⁵ |
| 100-100 | 20.2 ± 3.2 × 10⁻⁵ | 12.6 ± 2.6 × 10⁻⁵ | 8 ± 0.6 × 10⁻⁵ | 1.5 ± 0.6 × 10⁻⁵ |
| 100-50 | 16.5 ± 1 × 10⁻⁵ | 10 ± 0.7 × 10⁻⁵ | 6.3 ± 0.5 × 10⁻⁵ | 1.3 ± 0.2 × 10⁻⁵ |
| 50-100 | 16.3 ± 3.2 × 10⁻⁵ | 9.1 ± 1.6 × 10⁻⁵ | 6.5 ± 1.3 × 10⁻⁵ | 1.2 ± 0.3 × 10⁻⁵ |

Notably, sample 100-100 had the highest antibody and tPA content, as compared to samples 50-100 and 100-50. Further studies are needed to determine factors responsible for effect of the composition on the fibrinolytic activity of the conjugates.

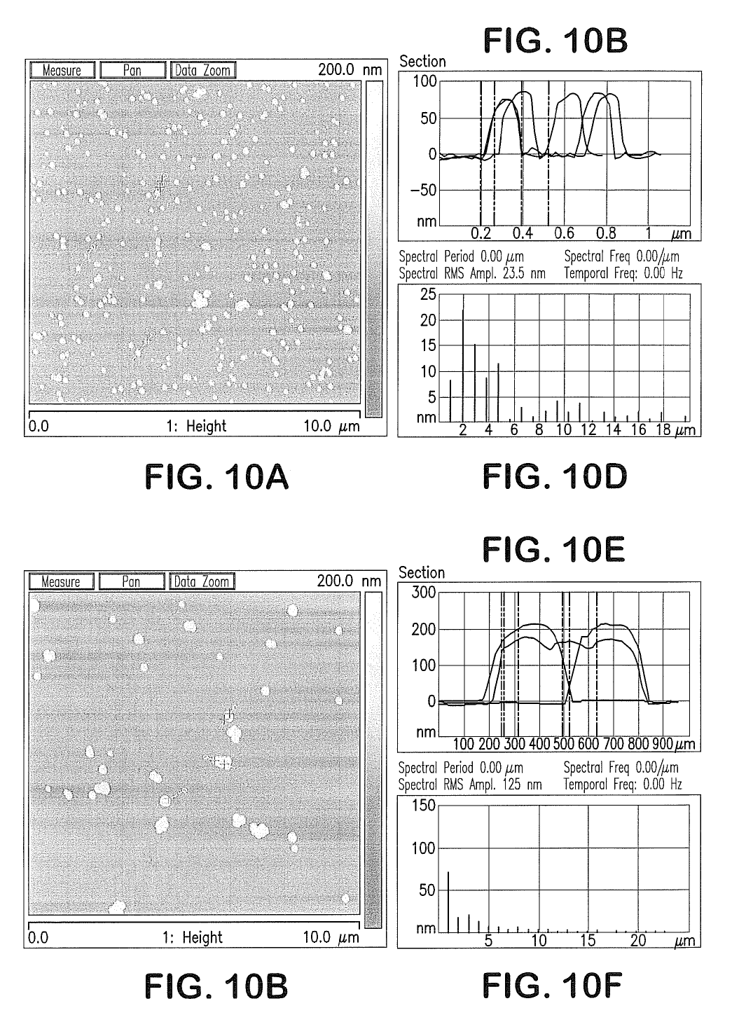

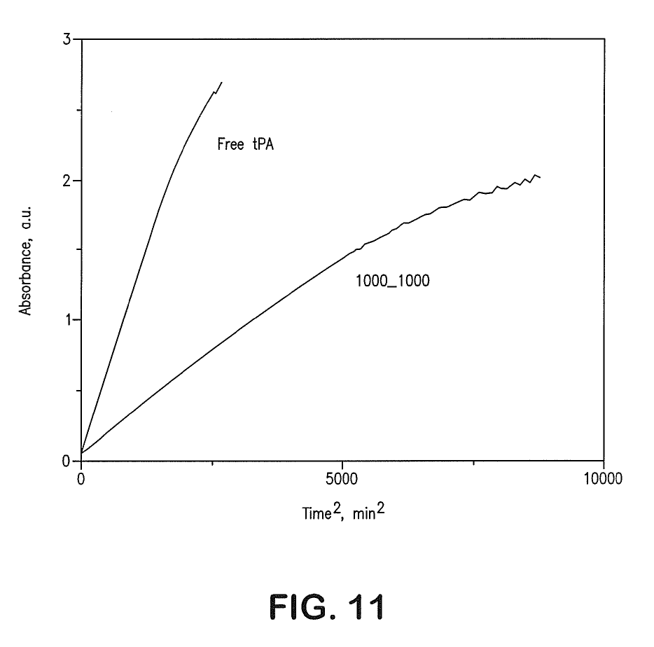

Interestingly, activity of protein-nanoparticle conjugates in clot lysis assay was either similar or only slightly lower than that of free tPA (FIG. 11), in spite of the fact that its activity in the absence of fibrin clots was more than three times lower that that of free tPA (FIG. 10). This property of protein-nanoparticle conjugates can be extremely important for therapeutic applications, because even if one disregards its targeted delivery to fibrin clot after injection, IV injection of the same dose of tPA-AF-nanoparticles, when compared to IV injection of free tPA can be expected to dissolve clots with approximately the same rate as free tPA (as modeled by clot lysis assay), but would cause much less cleavage of proteins in the circulation due to approximately 3-fold lower activity towards plasminogen in the absence of the clots. Analogous behavior is observed for tPA and AF attached to biodegradable polylactide nanoparticles.

### Example 3

Carboxyl-modified polylactic acid (PLA) nanoparticles having a size of 100 nm were functionalized with a NH2-PEG-COOH spacer. Following which, tPA and AF were coupled to the carboxyl group of the spacer. Both coupling reactions utilized standard carbodiimide chemistry. The surface of the nanoparticles was blocked with Tween® 20.

An initial mixture of in solution of 1000:1000 AF:tPA molecules (Solution 1000:1000) was found to yield 88:241 AF:tPA on the conjugated particle. For an initial mixture in solution of 500:500 AF:tPA molecules (Solution 500:500) was found to yield 41:120 AF:tPA on the conjugated particles.

FIG. 10 illustrates AFM images of the monodisperse PLA microbeads before (FIG. 10A) and after (FIG. 10B) covalent attachment of the tPA and the AF antibody. Cross-sections in the upper left corners of the images show the height of the particles (FIGS. 10C-10F).

Fibrin clots were prepared in microplate wells and the activity of the conjugated particles was evaluated as described in Example 2, above. The activity of the tPA conjugates in the absence of fibrin clots was measured by monitoring the reaction of a soluble chromogenic substrate S-2251 with plasmin produced from tPA cleavage of plasminogen. In this assay the activity was calculated as the initial slope of the kinetic curve linearized in coordinates [absorbance]−[time]². Results for are shown in FIG. 11. As can be seen, the activity of conjugated tPA was less than that of free tPA.

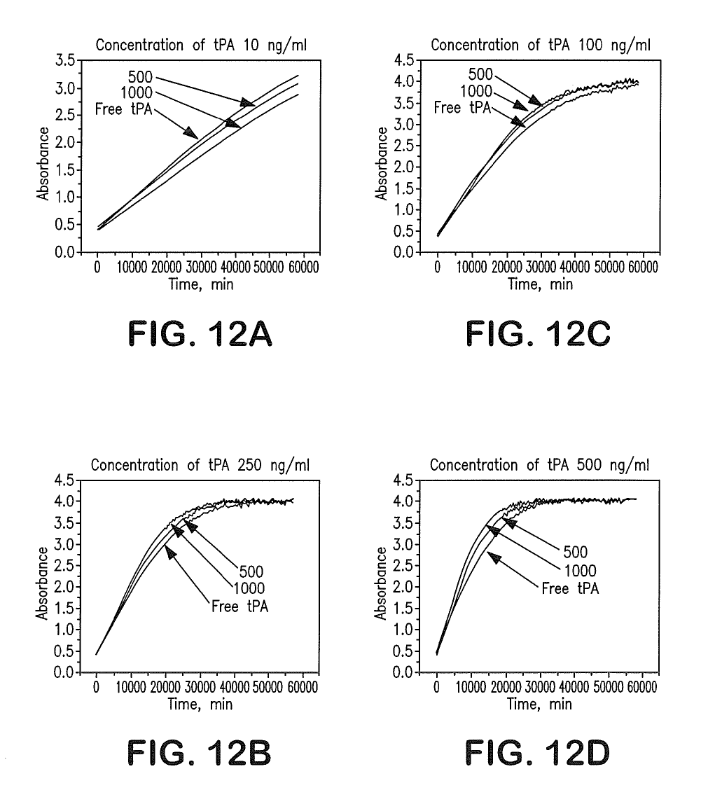

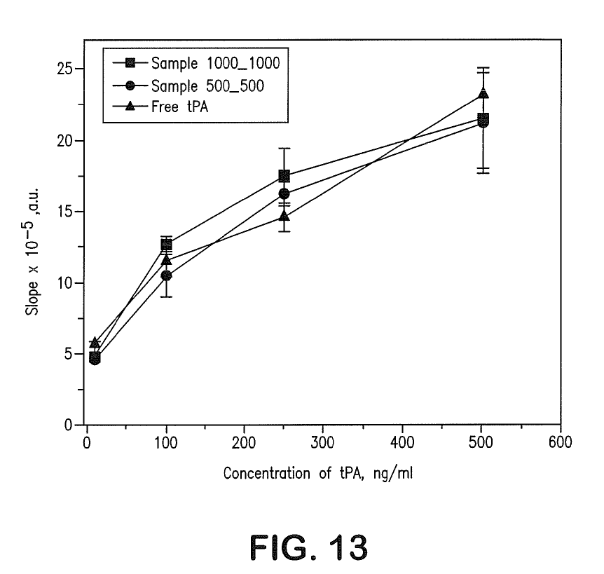

Additional kinetic data is shown in FIGS. 12A-D, which compare the clot dissolution rates of free tPA and the Solution 500:500 and Solution 1000:1000 conjugated particles over time. FIG. 13 illustrates the variation in clot dissolution rates for free tPA and the Solution 500:500 and Solution 1000:1000 conjugated particles versus tPA concentration. As can be seen, despite the lower activity when tPA is conjugated to PLA carrier particles, there is little difference in the clot dissolution rate.

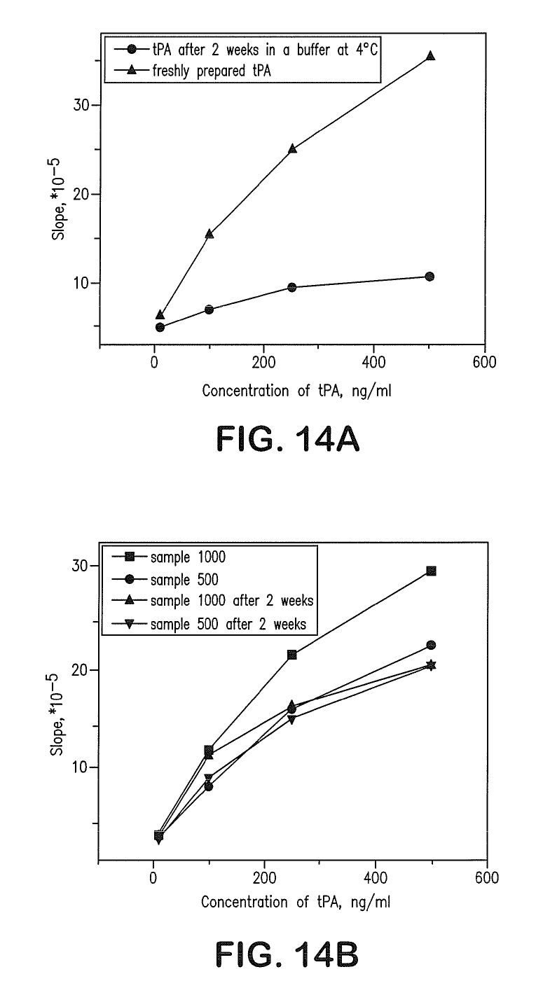

The stability of tPA-PLA-AF particles was examined over time. Results are shown in FIG. 14. As can be seen, after two weeks free tPA lost almost all of its activity (FIG. 14A). In contrast, the conjugated particles retained most of their activity over time (FIG. 14B).

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure as defined in the following claims and all equivalents thereto. Further, it is recognized that many embodiments may be conceived that do not achieve all of the advantages of some embodiments, yet the absence of a particular advantage shall not be construed to necessarily mean that such an embodiment is outside the scope of the presently disclosed subject matter.

What is claimed is:

1. A conjugated particle comprising:
a carrier particle defining a diameter of less than about 1000 micrometers;
a first polypeptide attached to the carrier particle, the first polypeptide being a fibrinolytic agent; and
a second polypeptide attached to the carrier particle, the second polypeptide specifically binding a component of a blood clot.

2. The conjugated particle of claim 1, wherein the carrier particle is less than about 50 nanometers in diameter.

3. The conjugated particle of claim 1, wherein the fibrinolytic agent catalyzes the activation of a substrate, wherein the active form of the substrate degrades fibrin.

4. The conjugated particle of claim 3, wherein the fibrinolytic agent is tissue plasminogen activator.

5. The conjugated particle of claim 1, wherein the fibrinolytic agent is an inactive form of a substrate, wherein the active form of the substrate degrades fibrin.

6. The conjugated particle of claim 5, wherein the fibrinolytic agent is plasminogen.

7. The conjugated particle of claim 1, further comprising a third polypeptide, the third polypeptide being a different fibrinolytic agent.

8. The conjugated particle of claim 1, wherein the second polypeptide specifically binds fibrin.

9. The conjugated particle of claim 8, wherein the second polypeptide is antifibrin antibody.

10. The conjugated particle of claim 1, further comprising a fourth polypeptide, the fourth polypeptide specifically binding a different component of a blood clot.

11. The conjugated particle of claim 1, wherein at least one of the first polypeptide and the second polypeptide is indirectly bound to the carrier particle with a spacer there between.

12. The conjugated particle of claim 11, wherein the at least one of the first polypeptide and the second polypeptide is covalently bound to the spacer, and the spacer is covalently bound to the carrier particle.

13. The conjugated particle of claim 11, wherein the spacer is a polymeric spacer.

14. The conjugated particle of claim 1, wherein the carrier particle comprises a polymer.

15. The conjugated particle of claim 1, wherein the carrier particle is biodegradable.

16. The conjugated particle of claim 1, wherein the carrier particle is homogeneous across the particle material.

17. The conjugated particle of claim 1, wherein the carrier particle is solid.

18. A method of dissolving a blood clot, the method comprising:
binding a conjugated particle to a component of the blood clot, the conjugated particle comprising a carrier particle defining a diameter of less than about 1000 micrometers, a first polypeptide attached to the carrier particle, and a second polypeptide attached to the carrier particle, the first polypeptide being a fibrinolytic agent, the second polypeptide specifically binding a component of the blood clot; and
degrading the fibrin in the blood clot according to the action of the fibrinolytic agent.

19. The method according to claim 18, wherein the component is fibrin.

20. The method according to claim 18, further comprising associating the bound conjugated particle with a substrate, wherein the first polypeptide activates the substrate.

21. The method according to claim 20, wherein the substrate is bound to a second conjugated particle.

22. The method according to claim 20, wherein the activated substrate degrades the fibrin.

23. The method according to claim 20, wherein the substrate is in solution.

24. The method according to claim 23, wherein the solution comprises plasma or whole blood.

25. The method according to claim 23, wherein the blood clot is in vivo.

26. The method according to claim 20, wherein the substrate is plasminogen.

27. The method according to claim 18, wherein the first polypeptide is tissue plasminogen activator.

28. The method according to claim 18, wherein the second polypeptide is antifibrin antibody.

29. A method for forming a conjugated particle comprising:
binding a first polypeptide to a carrier particle, the first polypeptide being a fibrinolytic agent; and
binding a second polypeptide to the carrier particle, the second polypeptide specifically binding a component of a blood clot.

30. The method according to claim 29, further comprising binding a spacer between the carrier particle and at least one of the first polypeptide and the second polypeptide.

31. The method according to claim 29, wherein the first polypeptide and the second polypeptide are simultaneously bound to the carrier particle.

32. The method according to claim 29, wherein the first polypeptide is bound to the carrier particle at a pH of about 6.

33. The method according to claim 29, wherein the first polypeptide and the second polypeptide are covalently bound to the carrier particle.

34. The method according to claim 29, wherein the first polypeptide is tissue plasminogen activator.

35. The method according to claim 29, wherein the second polypeptide is an antifibrin antibody.
